(19) United States
(12) Patent Application Publication
(10) Pub. No.: US 2020/0181558 A1
(43) Pub. Date: Jun. 11, 2020

(54) Multi-Needle Cell Seeding Device

(71) Applicant: Clemson University, Clemson, SC (US)

(72) Inventors: Dan Simionescu, Clemson, SC (US); Benjamin Jesse Fisher, Clemson, SC (US); Harrison Smallwood, Clemson, SC (US)

(21) Appl. No.: 16/669,702

(22) Filed: Oct. 31, 2019

Related U.S. Application Data
(60) Provisional application No. 62/753,177, filed on Oct. 31, 2018.

(51) Int. Cl. C12M 1/26; C12M 3/00

(52) U.S. Cl. CPC C12M 33/18; C12M 23/54

(57) ABSTRACT

Cell seeding devices and methods for utilization of the devices are described. The devices include a rolling needle support that carries a plurality of channeled needles thereon. Upon rolling the support over the surface of a scaffold, the needles penetrate the scaffold surface and deliver cells to the interior of the scaffold. The devices can deliver cells to a scaffold at a high density with high cellular retention and survival.

### CROSS-REFERENCE TO RELATED APPLICATIONS

The present application claims priority to U.S. Provisional Application Ser. No. 62/753,177, filed on Oct. 31, 2018, which is incorporated herein in its entirety by reference thereto.

### FEDERAL RESEARCH STATEMENT

This invention was made with Government support under Grant No. 1R56HL130950, awarded by the National Institutes of Health. The Government has certain rights in the invention.

### BACKGROUND

Regenerative medicine relates to treatment by use of cells (e.g., a patient's own cells, donor cells, stem cells, differentiated cells, etc.) to replace or regenerate diseased tissues or organs. To take advantage of regenerative cells, the cells are seeded via insertion or injection directly into diseased tissues or into scaffolds that can then be implanted. Scaffolds can be formed, for instance, of decellularized natural tissues or implantable synthetic materials. Regenerative medicine shows promise for treatment of any disease state in which tissue could be beneficially replaced or regenerated. Such diseases can affect any biological system including cardiovascular (e.g., atherosclerosis, heart, valve, vascular), orthopedic (e.g., osteoarthritis, osteoporosis), nervous (e.g., amyotrophic lateral sclerosis (ALS), multiple sclerosis (MS), Parkinson's disease), muscular (e.g., atrophy, dystrophy), metabolic (e.g., diabetes, end-stage renal disease), connective tissue (e.g., Ehlers-Danlos, Marfan's Syndrome), and pulmonary (e.g., chronic obstructive pulmonary disease (COPD)). Regenerative medicine also shows great promise for tissue and organ damage due to trauma (accident, war, etc.) or congenital disease.

The key to successful regenerative treatment approaches is successful cell seeding on/in tissue or a scaffold. Current methods for cell seeding include static seeding, in which cells are pipetted onto a scaffold; rotational seeding, in which a spinner flask mixes the scaffold and the cells together; centrifugal seeding, in which a device equipped with a centrifuge spins cells from a center tube out to the surrounding tissue at high speeds; vacuum seeding, in which a vacuum force is used to pull cells down through the pores of a scaffold; magnetic cell seeding, in which magnetic particles are incorporated into cells; and perfusion reactor-based seeding, which mimics physiological conditions to maximize cell morphology and extracellular matrix (ECM) remodeling. Unfortunately, these methods are often limited to only surface seeding of a scaffold (static seeding, rotational seeding); limited as to type, size, and geometry of scaffold (centrifugal seeding, vacuum seeding); present toxicity or cell/scaffold damage issues (centrifugal seeding, magnetic seeding); or are quite complex and expensive (perfusion reactor seeding).

Moreover, while known methods can work well for seeding gels and porous scaffolds, they are not equally successful for seeding denser scaffolds, such as fibrous tissue. Attempts to seed fibrous scaffolds or tissues for tissue regeneration have employed single syringe needles, surface seeding, or catheters with very low seeding efficacy. Unfortunately, these approaches are inefficient, slow, and of limited success in formation of a viable seeded scaffold.

What are needed in the art are devices and methods for seeding living cells into a support scaffold, particularly bulk seeding living cells into a support scaffold. Devices and methods that can seed cells into the interior of a scaffold (e.g., an implantable natural or synthetic scaffold or an in situ tissue scaffold) with high cell density and high success rate in an economical fashion would be of great benefit. Devices and methods that can also be used in cell therapy approaches, whereby healthy (stem) cells need to be delivered to diseased tissues to promote healing and regeneration.

### SUMMARY

According to one embodiment, disclosed is a cell seeding device that includes a handle and a needle support that is attachable to the handle. The needle support includes a cylindrical wall (e.g., a drum shape) that surrounds a central axis and has an inner surface and an outer surface. The needle support also defines a plurality of apertures through the cylindrical wall that extends from the inner surface to the outer surface. Upon attachment of the needle support to the handle, the cylindrical wall is capable of rotation about the axis. The device also includes a plurality of needles that projects outwardly from the outer surface of the needle support. Each needle includes a base that is at a first end of the needle at the junction of the needle and the outer surface and a tip that is at the opposite end of the needle. Each needle also defines a channel that is in fluid communication with at least one aperture of the needle support. The channel of each needle extends from the base of the needle toward the tip of the needle.

Also disclosed is a method for seeding a scaffold with a plurality of living cells. The method can include loading a fluid mixture, such as a cell suspension, into a reservoir of a cell seeding device, the mixture including living cells. The reservoir is in fluid communication with a plurality of needles that are carried on the outer surface of a needle support of the cell seeding device. The method also includes rolling the cylindrical wall of the needle support across a surface of the scaffold. As the support rolls across the scaffold, the needles penetrate an upper surface of the scaffold and the cells are delivered into the body of the scaffold via the fluid channels of the needles. Scaffolds can include ex vivo or in vitro cellular scaffolds, as well as in vivo cellular scaffolds that can be natural tissues or derived from natural tissues, synthetic materials, or some combination of both.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates one embodiment of a cell seeding device as described herein.

FIG. 2 schematically illustrates bulk cell seeding as may be carried out by a single needle of a device.

FIG. 3 illustrates an exploded view of a cell seeding device.

FIG. 4 illustrates a needle support of a cell seeding device.

FIG. 5 illustrates a side view and a top view of one embodiment of a needle as may be carried on a cell seeding device.

FIG. 6 illustrates a side view and a top view of another embodiment of a needle as may be carried on a cell seeding device.

FIG. 7 illustrates a side view and a top view of another embodiment of a needle as may be carried on a cell seeding device.

FIG. 8 illustrates a side view and a top view of another embodiment of a needle as may be carried on a cell seeding device.

FIG. 9 illustrates a side view and a top view of another embodiment of a needle as may be carried on a cell seeding device.

FIG. 10 illustrates one part of a separable handle of a cell seeding device.

FIG. 11 illustrates another part of a separable handle of a cell seeding device.

FIG. 12 illustrates a cut-away view of another embodiment of a cell seeding device.

FIG. 13 illustrates a perspective view of the cell seeding device of FIG. 12.

FIG. 14 illustrates fibrous acellular heart valve tissue utilized as a scaffold as described further herein.

FIG. 15 illustrates tissue of the scaffold following cell seeding with a device as described herein.

FIG. 16 represents one embodiment where living cells have been implanted within the fibers of a scaffold in accordance with the present disclosure.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

Disclosed are cell seeding devices and methods for utilization of the devices. The devices can deliver cells to a scaffold at a high density with high cellular retention and survival. Moreover, the devices can deliver a high density of cells in a homogenous fashion across a scaffold of any size and shape with little or no damage to the scaffold itself. Due to the efficient bulk cell delivery methods available by use of the devices, the seeding time for a scaffold can be reduced as compared to previously known devices, which can decrease costs and increase cell viability rates.

In one embodiment, disclosed devices can seed living cells within artificial or natural scaffolds in regenerative medicine study and treatment applications. Traditionally, cells have been either placed on top of scaffolds, creating multiple layers of superficial cells, or injected manually with a syringe inside the scaffold, generating large boluses of cells. Traditional processes have been supplemented by adding rotators, or subjecting scaffolds to vacuum or pressure. Similarly, attempts to deliver cells to diseased tissues for in vivo cell therapy typically utilizes injection of cells using a syringe or a catheter. However, none of these approaches can generate a homogeneous, high cell density construct with cells distributed in 3D that can be used as tissues for patients as can the disclosed devices.

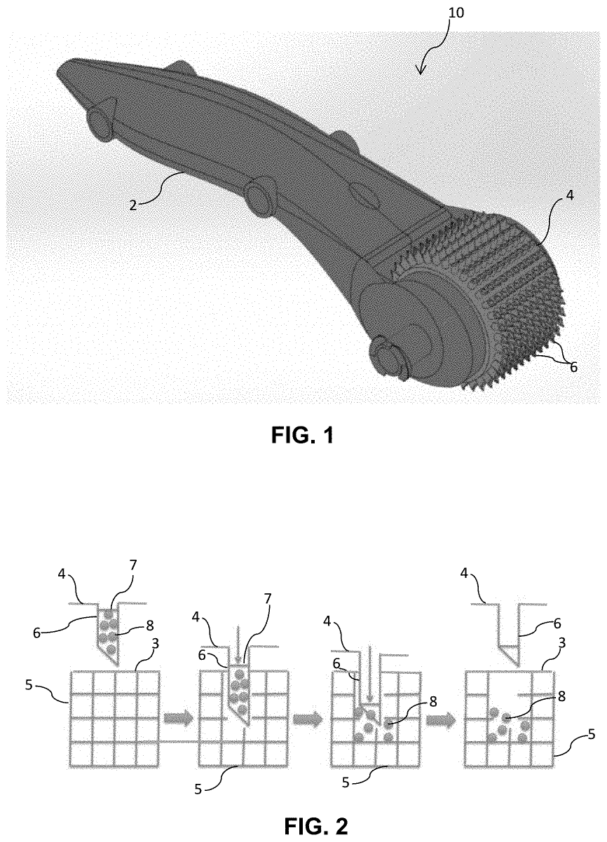

FIG. 1 illustrates one embodiment of a device. As shown, a bulk cell seeding device 10 includes a handle 2 and a rotatable needle support 4. The rotatable needle support 4 carries multiple needles 6 on its surface. As shown in FIG. 2, as the needle support 4 contacts the upper surface 3 of a scaffold 5, a needle 6 carried by the needle support 4 can penetrate the surface 3. Upon penetration, a mixture 7 that carries living cells 8 therein can be delivered via the needle 6 to the interior of the scaffold 5. Upon removal of the needle 6 from the scaffold 5 as the rotatable needle support 4 is rolled across the upper surface 3 of the scaffold 5, the mixture 7 and the cells 8 contained therein can remain within the interior of the scaffold 5.

As utilized herein, the term “scaffold” generally refers to any cellular support material, including synthetic materials, natural materials, modified natural materials, or some combination thereof. Decellularized natural tissues, as well as composite structures including natural tissues (or modified natural tissues) supported by synthetic materials, are also encompassed. Scaffolds can be ex vivo, in vitro, or in vivo during seeding. In one embodiment, scaffolds can be implantable materials and can be intended for implant following or prior to cell seeding by use of disclosed devices. In another embodiment, scaffolds can be intended for use in a laboratory or study application, and need not be designed for implant in a research or treatment protocol.

Disclosed devices can be utilized with scaffolds of any design. For instance, devices can be utilized to seed porous scaffolds such as sponges, that are relatively easy to seed with cells as the cells can readily diffuse into various pores and can distribute homogeneously. Beneficially, disclosed devices can also be successfully utilized in seeding more dense scaffolds such as dense tissues including, without limitation, arteries, valves, tendons, skin, and synthetic equivalents thereof. Such dense tissues have proven difficult or impossible to regenerate by simply seeding cells on a surface thereof or by injection of a bolus of cells by use of a syringe. Disclosed devices can seed cells into dense scaffolds or tissues at multiple locations with high efficacy so as to generate homogeneous, high cell density constructs with cells distributed in three dimensions.

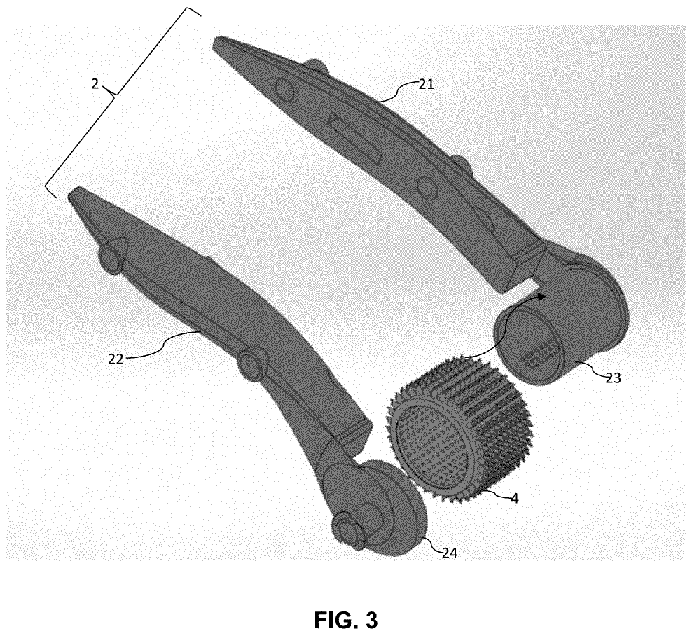

FIG. 3 illustrates the cell seeding device of FIG. 1 in an exploded view. As shown, in this embodiment, the handle 2 is separable into a first portion 21 and a second portion 22. The first portion 21 includes a reservoir 23 which, during use, can be charged or loaded with a mixture that carries the living cells for delivery. The second portion 22 includes a cap 24 that can cap and seal the reservoir 23 and prevent leakage of the charged mixture. During use, the needle support 4 can be located (arrow) on the reservoir with suitable tolerance so as to be capable of rotation around the reservoir 23, and the cap 24 can cap the reservoir 23 when the portions 21, 22 are attached to one another to form the unitary handle 2.

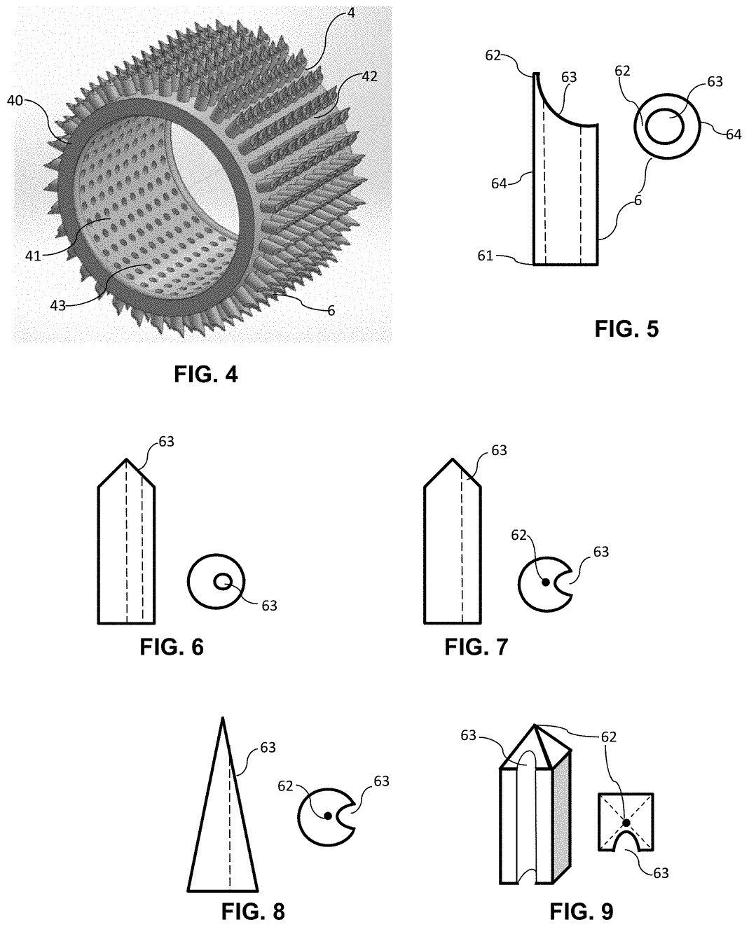

FIG. 4 provides a perspective view of the needle support 4 in greater detail. As shown, the needle support 4 includes a cylindrical wall 40 defined by an inner surface 41 and an outer surface 42. The needle support 4 also includes a plurality of apertures 43 that pass completely through the wall 40 from the inner surface 41 to the outer surface 43. As such, a fluid contacting the inner wall 41 of the needle support 4 can pass through the apertures 43.

A plurality of needles 6 are carried on the outer surface 42 of the needle support. FIG. 5 presents a side view (left) and a top view (right) of a single needle 6 as may be carried on the outer surface 42 of a needle support 4. As shown, each needle typically includes a base 61, a tip 62, and an exterior surface 64. As shown in FIG. 4, the base 61 is the portion of the needle 6 that is proximate to the outer surface 42 of the needle support 4. The tip 62 of the needle 6 is the point of the needle 6 that is furthest from the base 61. Although the tip 62 may be variously formed, it typically has a radius that is less than or equal to about 1 micrometer.

Each needle 6 contains at least one channel 63. The channel 63 may be located in a variety of different positions, such as completely in the interior of the needle 6, on an exterior surface 64, etc. In the embodiment illustrated in FIGS. 4-5, for example, the channel 63 is located in the interior of the needle 6, forming a lumen that extends from the base 61 toward the tip 62 of the needle 6. The cross-section of the channel 63, can be substantially circular, as shown. The channel 63 may also be generally U-shaped, arcuate, or have any other configuration suitable for moving a substance therethrough, such as, for example, V-shaped or C-shaped.

Each needle 6 is located on the outer surface 42 of the needle support 4 such that the channel 63 is in fluid communication with at least one aperture 43 that passes through the cylindrical wall 40. As such, a pathway is formed by a channel 63 and an aperture 43, which meet at a junction that is generally located at the meeting of the outer surface 42 and the needle base 61. Each needle support may deliver cells along the plurality of pathways, each pathway being formed for each of the needles 6. Each pathway enables living cells to flow from the inner surface 41 through the aperture 43, and into the channel 63 of a needle.

The needles 6 of a device can vary. For example, FIG. 6-FIG. 9 illustrate alternative embodiments for individual needles 6 of a device, though the needles of a device are in no way intended to be limited to these particular embodiments. By way of example, the channel 63 may be variously positioned on the exterior surface 64 of a needle, forming a substantially linear path from the base 61 towards the tip 62, or alternatively, forming a winding or circuitous path along the exterior surface 64. In addition, the channel 63 may be completely surrounded by the wall of the needle 6, as in FIG. 5 and FIG. 6, or may form an open channel on a surface of the needle, as in FIG. 7-FIG. 9. Optionally, a needle may include multiple channels; where two or more channels are present on a needle, the channels 63 may be variously spaced around the needle 6 in a symmetrical or asymmetrical manner.

The shape of the exterior surface 64 of the needles can vary. For instance, a needle can have a generally cylindrical body topped with a tip as in FIG. 5-FIG. 7. In some embodiments, the cylindrical body can be topped with a conical portion that includes the needle tip, as in FIG. 6 and FIG. 7. However, other needle shapes are encompassed, such as conical needles (FIG. 8), and needles in which all or a portion of the needle is pyramidal (FIG. 9).

The size of the channel 63 can vary, generally depending upon the size of the cells to be delivered by use of a device. For example, in some embodiments, the cross-sectional dimension of the channel typically ranges from about 100 micrometers to about 5 centimeters, for instance from about 200 micrometers to about 1 millimeter, in some embodiments. The dimension of a single channel 63 may be constant or it may vary as a function of the length of the channel. The length of the channel may also vary to accommodate different volumes, flow rates, and dwell times for the cell mixture. The cross-sectional area of the channel may also vary. The dimensions of a channel 63 can be specifically selected in one embodiment to induce capillary flow of a cell mixture. Capillary flow generally occurs when the adhesive forces of a fluid to the walls of a channel are greater than the cohesive forces between the liquid molecules.

In addition to seeding cells, the device and method of the present disclosure can be used in various other applications. For instance, the device can be used to deliver drugs or other agents to selected areas. The device can also be used to introduce air into scaffolds to increase porosity.

It should also be understood that the number of needles 6 shown in the figures is for illustrative purposes only. The actual number of needles used in the cell seeding device may, for example, range from about 50 to about 1,000, or from about 100 to about 500, in some embodiments.

The area of the apertures 43 may vary and will depend on factors such as, for example, the diameter of the needles 6, the viscosity of the cell mixture to be moved through the pathway, and the quantity of substance to be delivered. In certain embodiments, the cross-sectional dimensions of the apertures 43 can be equal to that of a channel 63 that aligns with the aperture, although smaller dimensions may also be acceptable for use in the present invention. In selected embodiments, an aperture 43 and channel 63 have sides that are coextensive with each other. Regardless of their particular configuration, however, the needles 6 define at least one channel 63 that is in fluidic communication, with at least a portion of an aperture 43 of the needle support 4.

The needle support 4 may be constructed from metal, ceramic, plastic, or other material. The wall 40 of the needle support 4 can vary in thickness to meet the needs of the cell seeding device, such as about 0.5 millimeter or more, in some embodiments from about 1 to about 10 millimeters.

The needles are typically of a length sufficient to penetrate the surface of a cellular scaffold, but not sufficiently far into the scaffold to penetrate completely throughout the scaffold. In certain embodiments, the needles have a length (from their tip 62 to their base 61) of about 25 millimeters or less, and in some embodiments from 1 to about 10 millimeters. In addition, needles 6 can vary in length from one another on a single needle support 4. For instance, a needle support 4 can carry needles of two, three, four, or more different lengths that can be provided in a predetermined pattern or randomly over the outer surface 42 of the needle support 4. By use of such, cells of a cell mixture can be delivered to different depths throughout the total depth of a scaffold by simply rolling the needle support 4 across a surface of the scaffold.

The needles 6 may be arranged on the outer surface 42 in a variety of patterns, and such patterns may be designed for a particular use. For example, the needles may be spaced apart in a uniform manner, such as in a rectangular or square grid or in concentric circles. The spacing may depend on numerous factors, including height and width of the needles 6, as well as the amount and characteristics of cell mixture that is intended to be moved through the needles 6.

The needles 6 may be formed of various substances such as, for example, polymers, ceramics, and metals. In one embodiment, the needles 6 and the cylindrical wall 40 can be formed of the same material and unitary. While numerous processes may be used to manufacture a needle support 4 according to the present invention, a suitable production system is additive manufacturing technology, e.g., 3D printing. Additive manufacturing refers to any method for forming a three-dimensional object in which materials are deposited according to a controlled deposition and/or solidification process. The main differences between additive manufacturing processes are the types of materials to be deposited and the way the materials are deposited and solidified. Some methods extrude materials including liquids (e.g., melts or gels) and extrudable solids (e.g., clays or ceramics) to produce a layer, followed by spontaneous or controlled curing of the extrudate in the desired pattern. Other processes deposit solids in the form of powders or thin films, followed by the application of energy and/or binders often in a focused pattern, to join the deposited solids and form a single, solid structure having the desired shape. In some methods, successive layers are individually treated to solidify the deposited material prior to deposition of the succeeding layer, with each successive layer becoming adhered to the previous layer during the solidification process. A needle support 4, or portions thereof, can be formed of any suitable additive manufacturing process, with preferred methods generally depending upon the nature of the material to be used in forming the needle support.

Alternatively, the needles and apertures of a needle support 4 can be formed by a material removal process. For instance, a cylinder can be formed (e.g., molded) from a plastic or metal, and the needles and apertures can be subsequently formed by an etching process.

In any case, in general, materials of formation of a device can generally be sterilizable. All or components of the device can be disposable or reusable. For instance, in one embodiment, all components of a device can be separable and reusable following cleaning and sterilization. In other embodiments, a portion of the device, e.g., the reservoir and/or the needle support, can be disposable while other components, e.g., the handle, can be reusable.

As indicated above, the inner surface 41 of the needle support 4 of the cell seeding device can be in fluid communication with a reservoir that can initially retain a cell mixture. The term “reservoir” generally refers to a designated area or chamber configured to retain a fluidic mixture. The reservoir may be an open volume space, gel, solid structure, etc. In most embodiments, the reservoir defines an open volume space through which a cell mixture is capable of flowing to contact the inner surface 41 of the cylindrical wall 40.

In one embodiment, the interior volume defined by the cylindrical wall 40 can define the reservoir. In other embodiments, the cell seeding device can include a separable reservoir that can be placed in fluid communication with the interior surface 41 of the needle support 4 during use. For instance, in one embodiment, the handle of a device can incorporate a reservoir. In other embodiments, the reservoir can be an exterior container that can be placed in fluid communication with the inner surface 41 of the needle support 4 during use, e.g., via connected tubing or the like.

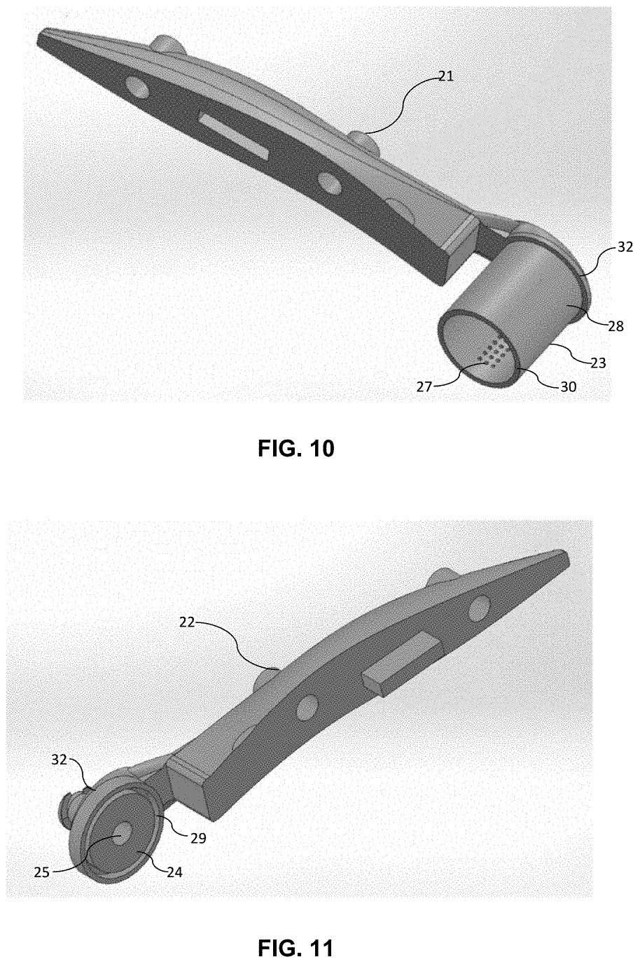

FIG. 10 and FIG. 11 illustrate sections 21 and 22, respectively, of a handle 2 of a device. As shown, in this embodiment, section 21 can include a reservoir 23, and the section 22 can include a cap 24 that can fit and close the reservoir 23 upon assembly of the device.

In one embodiment, the reservoir 23 can be filled with a cell mixture prior to assembly of a cell seeding device. In another embodiment, the reservoir 23 or the cap 24 can include a fitting or port 25 allowing for a cell mixture to be infused into the reservoir 23 following assembly using a syringe or other fluid delivery device.

A cell mixture can include any desired type of cell (or combination thereof) suspended in a fluid. For instance, and without limitation, a fluid can include saline, standard intravenous solutions, cell culture medium, or other cell nutrient solutions, serum, plasma, or blood, with or without inert additives, such as dextran to increase viscosity or more complex additives such as fibrinogen or fluid polymers.

Cells can be incorporated in the cell mixture at any desired concentration, provided that the cell mixture is capable of flowing through the apertures and needles for successful delivery by use of the device. For instance, the viscosity of a cell mixture can vary from that of water (about 1 centipoise at 20° C.) to that of blood (about 10 centipoise at 20° C.), or even higher in some embodiments. Similarly, a cell mixture can incorporate cells at any desired concentration level, for instance from about 100 cells/mL to about 10⁶ cell/mL, or even higher in some embodiments.

The reservoir 23 can be in fluid communication with the inner surface 41 of the needle support 4 upon assembly. For instance, the reservoir 23 can include one or more apertures through which a cell mixture can flow during use. Apertures 27 can be of any size and shape. For instance, apertures 27 can align with the channels 63 of the needles 6 as the needle support 4 rotates around the reservoir 23 during use, allowing the cell mixture to flow from the reservoir 23 through the apertures 27 of the reservoir, through the apertures 43 of the needle support 4 and through the channels 63 of the needles 6. Alternatively, apertures 27 can be larger or smaller than the apertures 43 of the needle support 4. By way of example, reservoir 23 can define one or more relatively large apertures 27 therethrough (e.g., in the form of slits or ovals). The outer surface 28 of the reservoir 23 and/or the inner surface 41 of the needle support 4 can also define a raised edge so as to provide an open space between the two upon assembly. During use, that open space can carry an amount of the cell mixture for delivery through the needles 6 and can be continuously replenished from the cell mixture carried in the reservoir 23.

The cap 24 can fit over an open end of the reservoir 23 upon assembly of the two portions 21, 22. For instance, the cap 24 can define a groove 29 therein that can mate with the end 30 of the reservoir 23, optionally with a gasket (not shown) therein, to close and seal the reservoir 23 upon assembly.

Upon assembly (FIG. 1) and loading of a cell mixture into the reservoir, upon pushing or pulling the handle 2 with the outer surface 42 of the needle support engaged with the surface of a scaffold, the needle support can rotate about the axis and roll across the surface of the scaffold. Pressure can be applied over the scaffold surface such that the needles 6 can pierce the surface and deliver cell mixture to the interior of the scaffold via the multiple needle tips 62.

In one embodiment, flow of cell mixture out of the reservoir can be enabled by the initial filling of the reservoir with the cell mixture and capillary flow through the various apertures and the needle channels. In such an embodiment, a device can generally also include an air inlet (e.g., at port 25) to prevent vacuum and loss of flow. In other embodiments, the reservoir can be pressurized to provide a continuous force on the cell mixture to encourage flow out of the reservoir and the needles. For instance, in one embodiment, the reservoir 23 can be in fluid contact with a pressurized gas source, e.g., a balloon, a gas source, or the like, to provide flow pressure as needed.

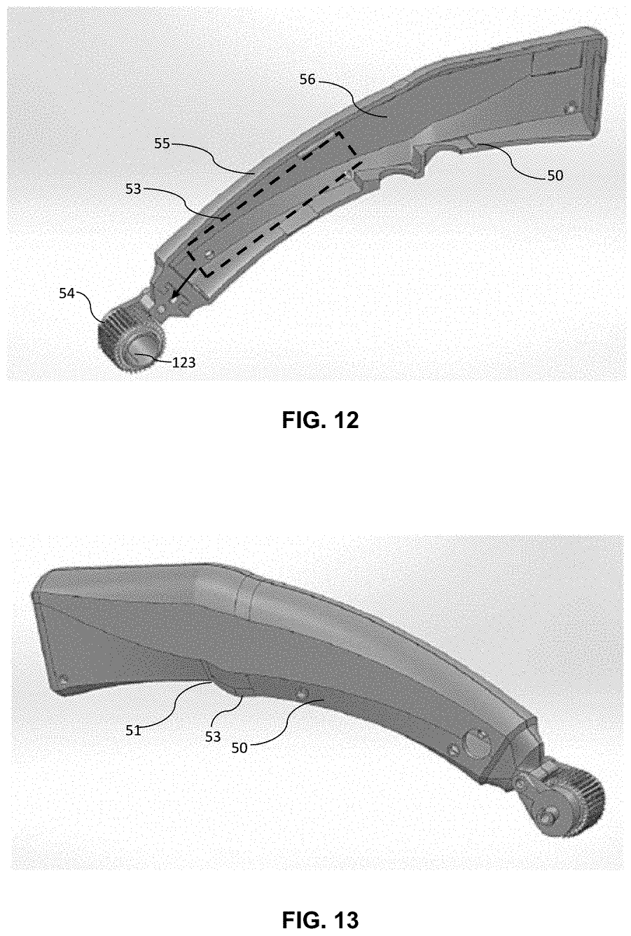

In one embodiment, the cell seeding device can carry a pressure source. For instance, FIG. 12 and FIG. 13 illustrate another embodiment of a device 50 that can carry an air pump (e.g., a battery-operated air pump) 53 within the handle 55, for instance within an inset 56 within the handle 55. The device 50 can be configured such that the pump 53 carried within the inset 56 can be placed in communication with a needle support 54 (e.g., via arrow), and in suitable communication between a pump 53 carried in the handle 55 and a reservoir 123 within the needle support 54. FIG. 13 illustrates the device 50 in perspective. A device can include additional features, such as one or more finger grips 51 for improved handling, an on/off switch 53 for an air pump, etc.

Thus, either by use of pressure increase in the internal reservoir by use of a pressurized gas or by merely equilibration with surrounding atmosphere combined with capillary flow, upon rolling the needle support of a device across a scaffold surface, a cell mixture can be delivered from the reservoir through the needles and into the interior of the scaffold.

The pressure applied to the surface of a tissue can be controlled either automatically or manually. For instance, in one embodiment, and referring again to FIG. 10, the handle can include one or more ridges 32 that, upon assembly of the device, can extend radially outward to a distance that is greater than the length of the needles of the needle support. Thus, upon rolling the needle support over the surface of a scaffold, the ridges 32 can prevent excessive force on the scaffold by the needle support and can prevent the needles from penetrating the scaffold.

In one embodiment, pressure applied to the surface of a scaffold can be controlled by inclusion of a pressure sensor on the device. For instance, the device can carry a pressure sensor that can sense the pressure applied as the needle support 54 is rolled over a surface. A pressure sensor can optionally include a visible readout, e.g., on the handle. Thus, a user can monitor the pressure applied to the surface of a scaffold and can manually adjust the applied pressure as necessary so as to control the depth of cell delivery within the tissue.

A cell seeding device can be operated manually or can be integrated within an automated system that controls pressures, rolling direction, speed, or force applied for a single or multiple scaffold(s) or tissue(s). The device can provide homogeneous cell seeding by delivering a controlled volume of cell suspension via the rotatable needle support, which concomitantly delivers cells through the channeled needles.

Devices can be rolled over the surface of a single-cell scaffold multiple times without damage to the scaffold. This can provide a large number of individual cell insertions to a single scaffold. For instance, a needle support can be rolled over a scaffold surface in multiple different directions for multiple cycles with the result of injecting a small volume of cells in hundreds or thousands of different locations within the scaffold.

Through use of the cell seeding device, cells can be seeded at high density within tissues or scaffolds, including dense and fibrous scaffolds such as arteries, veins, valves, myocardium, tendons, ligaments, etc. Such capabilities can provide a successful route to seeding scaffolding of a wider variety of characteristics. For instance, as technologies are available to generate a variety of scaffolds, scaffolds that match desired biological and mechanical properties of target tissues can be prepared and then seeded by use of disclosed devices, without the need to compromise scaffold design in order to obtain desired cell seeding density and viability.

Disclosed devices can be utilized in tissue regeneration by seeding large number of cells in desired locations and densities in a homogeneous pattern and can also allow for more efficient delivery of cells to tissues of interest for the purpose of cell therapy. For example, decellularized heart valves, arteries, veins, nerves, ureters, muscle tissue, tendons, and ligaments (CryoLife, LifeNet Health, Humacyte) can be seeded with patient-provided cells and then implanted for regeneration of tissue by a subject's own cells, decreasing likelihood of side effects due to implantation of foreign materials.

In one embodiment, disclosed devices can be utilized in heart valve replacements. Current valve substitutes provide excellent quality of life but have limited durability, mostly because these are non-living implants. The most physiologic valve replacements are considered the valve homografts, which have excellent hemodynamics but lack cells. An ideal valve substitute would be an in vitro regenerated, living construct which closely mimics the unique biological and hemodynamic features of the aortic root. The aortic root comprises distinct anatomical components, extracellular matrix molecules and cells, which maintain a balanced matrix homeostasis; the secret to life-long mechanical endurance. By use of disclosed devices, functional aortic root regeneration in vitro is possible by seeding each anatomical component of xenogeneic acellular roots with adult stem cells or pre-differentiated adult stem cells, and exposing cell-seeded roots to controlled mechanical cues to induce stem cell differentiation and maturation into target cells as a response to the “niche” biology and biomechanics.

The present disclosure may be better understood with reference to the Examples set forth below.

### EXAMPLES

### Example No. 1

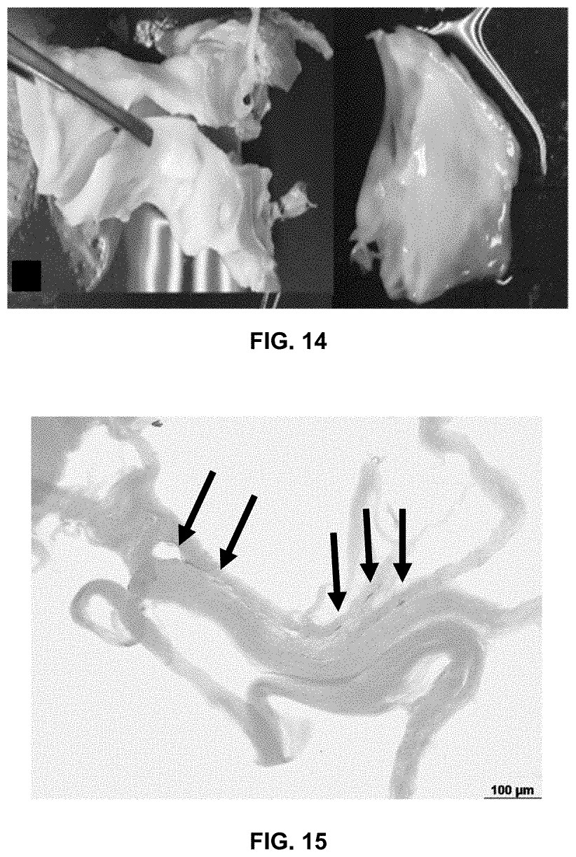

A fibrous acellular valve scaffold was prepared (FIG. 14). Stem cells that were cultured in vitro were loaded into a device as illustrated in FIG. 1. The device included 350 needles on the surface of the needle support. The device was rolled 5 times radially across the scaffold and 5 times circumferentially around the scaffold. Following, the scaffold was sectioned, and tissues stained with hematoxylin and eosin. Results are shown in FIG. 15. The black arrows point to living cells implanted within the fibers of the scaffold.

### Example No. 2

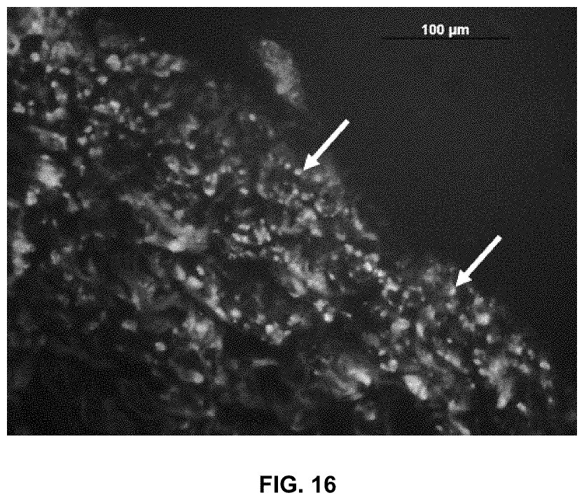

A myocardial acellular scaffold was prepared by removing all cells from a whole porcine heart using detergents and enzymes. Human fibroblasts that were cultured in vitro were loaded into a device as illustrated in FIG. 1. The device included 350 needles on the surface of the needle support. The device was rolled across the scaffold and 5 times circumferentially around the scaffold. Following, the scaffold was sectioned, and tissues stained with DAPI, which stains nuclei bright fluorescent. Results are shown in FIG. 16. The white arrows point to living cells implanted within the fibers of the scaffold.

While certain embodiments of the disclosed subject matter have been described using specific terms, such description is for illustrative purposes only, and it is to be understood that changes and variations may be made without departing from the spirit or scope of the subject matter.

What is claimed is:

1. A cell seeding device comprising:
a handle;
a needle support that is attachable to the handle, the needle support comprising a cylindrical wall having an inner surface and an outer surface, the cylindrical wall surrounding an axis, the needle support comprising a plurality of apertures through the cylindrical wall and extending from the inner surface to the outer surface, wherein upon attachment to the needle support to the handle, the cylindrical wall is capable of rotation about the axis;
a plurality of needles projecting outwardly from the outer surface of the cylindrical wall, each needle comprising a base at a first end of the needle and a tip at an opposite end of the needle, the base meeting the outer surface of the cylindrical wall at a junction, each needle defining a channel that is in fluid communication with an aperture of the needle support, the channel extending from the base of the needle toward the tip of the needle; and
a reservoir in fluid communication with the inner surface of the needle support.

2. The device of claim 1, the handle comprising the reservoir.

3. The device of claim 2, the reservoir defining one or more apertures therethrough.

4. The device of claim 1, wherein the channel is completely surrounded by a wall of the needle.

5. The device of claim 1, wherein the channel is open at a surface of the needle.

6. The device of claim 1, the plurality of needles comprising needles of different lengths.

7. The device of claim 1, wherein the device is connectable to a pressurized gas source.

8. The device of claim 1, the handle further comprising an air pump.

9. The device of claim 1, wherein the reservoir is in fluid communication with the inner surface of the needle support via tubing.

10. A method for seeding a scaffold with a plurality of living cells, the method comprising:
loading a cell mixture into a reservoir, the cell mixture containing a fluid and one or more types of living cells suspended in the fluid;
rolling a cell seeding device across a surface of a scaffold, the cell seeding device including a needle support, the needle support comprising a cylindrical wall having an inner surface and an outer surface and defining an axis therethrough, the needle support comprising a plurality of apertures through the cylindrical wall and extending from the inner surface to the outer surface, the inner surface of the cylindrical wall being in fluid communication with the reservoir, a plurality of needles projecting outwardly from the outer surface of the cylindrical wall, each needle comprising a base at a first end of the needle and a tip at an opposite end of the needle, the base meeting the outer surface of the cylindrical wall at a junction, each needle defining a channel that is in fluid communication with an aperture of the needle support; wherein
upon the rolling of the cell seeding device across the surface of the scaffold, a portion of the cell mixture passes out of the channel of one of the needles and is delivered into the interior of the scaffold beneath the surface of the scaffold.

11. The method of claim 10, wherein the cell mixture comprises stem cells.

12. The method of claim 10, wherein the cell mixture comprises ex vivo cells.

13. The method of claim 10, wherein the scaffold comprises natural tissue.

14. The method of claim 13, wherein the natural tissue comprises decellularized natural tissue.

15. The method of claim 10, wherein the scaffold comprises a fibrous construct.

16. The method of claim 10, wherein the scaffold comprises synthetic materials.

17. The method of claim 10, further comprising placing the reservoir under increased pressure to encourage passage of the cell mixture through the channel.

18. The method of claim 10, wherein the method is automated.

19. The method of claim 10, comprising rolling the cell seeding device across the surface of the scaffold multiple times.
